(19) United States
(12) Patent Application Publication
(10) Pub. No.: US 2018/0177960 A1
(43) Pub. Date: Jun. 28, 2018

(54) SENSING ARRANGEMENT FOR GAS DELIVERY SYSTEM

(71) Applicant: FISHER & PAYKEL HEALTHCARE LIMITED, Auckland, (NZ)

(72) Inventor: Callum James Thomas SPENCE, Auckland (NZ)

(21) Appl. No.: 15/127,365

(22) PCT Filed: Mar. 20, 2015
(86) PCT No.: PCT/NZ2015/050033

§ 371 Date: Sep. 19, 2016

Related U.S. Application Data
(60) Provisional application No. 61/968,507, filed on Mar. 21, 2014.

(51) Int. Cl. A61M 16/00; A61M 16/16; A61M 16/06; A61M 16/08

(52) U.S. Cl. CPC A61M 16/024; A61M 16/0066; A61M 16/16; A61M 16/0666; A61M 16/0051; A61M 16/0003; A61M 2206/20; A61M 2016/0027; A61M 2205/502; A61M 2016/0039; A61M 16/161; A61M 16/085; A61M 16/0858

(57) ABSTRACT

A gas delivery system adapted to deliver a gas to a subject may have a gases flow passage with a modulation section where the velocity of gases passing through the modulation section is modulated. The modulation section can have a first region at which the modulation of the velocity of the gases is greatest. The gases flow passage can be configured in such a way that the pressure measured at or near the first region is substantially the same as the static pressure of gases at or near a second region along the gases flow passage downstream of the first region.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### TECHNICAL FIELD

The present disclosure generally relates to respiratory therapy devices. More particularly, the present disclosure relates to sensing arrangements for use with respiratory therapy devices.

### BACKGROUND

A patient dealing with respiratory illness, for example, chronic obstructive pulmonary disease (COPD), can have difficulty effecting a sufficient exchange of gases with his or her environment. This difficulty may be the result of a variety of physiological faults, including a breakdown of lung tissue, dysfunctions of the small airways, excessive accumulation of sputum, or cardiac insufficiency. With such illnesses, it is useful to provide the patient with a therapy that can improve the ventilation of the patient. In some situations, the patient can be provided with a respiratory therapy system that includes a gas source, an interface that may be used to transmit gas to the airway of a patient, and a conduit extending between the gas source and the interface. The gas source may, for example, be a container of air and/or another gas suitable for inspiration, e.g. oxygen or nitric oxide, a mechanical blower capable of propelling a gas through the conduit to the interface, or some combination of both. The respiratory therapy system may include a means or arrangement for heating and/or humidifying gases passing through the system to improve patient comfort and/or improve the prognosis of the patient's respiratory illness.

### SUMMARY

It is useful for a respiratory therapy system to be capable of measuring or estimating characteristics of the gases flowing through the system at various locations along the system. For example, it may be useful to know, for example, the flow rate, pressure, absolute humidity, relative humidity, O2 concentration and/or CO2 concentration of gases flowing through the system. These characteristics may be relayed to a user of the system through an output device that may deliver visual and/or audial information to the user. In some configurations, the characteristics may be used to help control various aspects of the operation of the system.

As mentioned above, it is useful to know the pressure of gases flowing through the respiratory therapy system. For example, it may be useful to measure a pressure of delivered gases at or near an interface. If the measured pressure is too high, the gas therapy may cause discomfort and/or may indicate an improper interface fit, which may diminish the efficacy of the therapy. However, while it is useful to place the sensor as close as possible to the airway of the patient, it may be inconvenient to place the sensor at such a location. For example, there may not be enough free space in the interface to accommodate the sensor. Additionally, if the sensor is located in or on a patient interface, the cost of producing the interface may increase. Accordingly, certain features, aspects and advantages of the present disclosure provide an improved sensing arrangement that might solve one or more of the above problems, or at least provide the public with a useful choice.

Thus, in accordance with certain features, aspects and advantages of at least one of the embodiments disclosed herein, a gas delivery system is disclosed. The systems, methods and devices described herein have innovative aspects, no single one of which is indispensable or solely responsible for their desirable attributes. Without limiting the scope of the claims, some of the advantageous features will now be summarized.

The gas delivery system may be a respiratory therapy system. The gas delivery system may comprise a flow generator adapted or configured to propel gases. The gas delivery system may comprise a gases flow passage. The gases flow passage may be adapted to receive gases from the flow generator and channel them to a subject. The subject may be a patient. The gas delivery system may comprise a patient interface, which can be an unsealed patient interface, such as a nasal cannula. The gas delivery system may comprise a sensing module. The sensing module may be configured to measure gas pressure, gas flow rate, and/or other characteristics of gases. The sensing module may be located inside or outside of the gases flow passage. The gases flow passage may comprise a modulation section. In use, the velocity of gases passing through the modulation section may be modulated. In some configurations, the velocity of such gases may increase. In some configurations, the velocity of such gases may decrease. The modulation section may comprise a first region at which the modulation of the velocity of the gases passing through the modulation section is greatest or at a maximum. The sensing module may measure the pressure, gas flow rate, and/or other characteristics of gases at or near the first region. The sensing module may be located at or near the first region. The gases flow passage may be configured such that, when the flow generator is propelling gases through the gases flow passage, the measured pressure of gases at or near the first region may be substantially the same as the static pressure of gases at or near a second region along the gases flow passage downstream of the first region.

The gases flow passage may comprise a wall. The wall may comprise a port. The port may be located at a part of the wall at or near the first region. The port may be sealed from the atmosphere outside of the port. The sensing module may be located in or on the port. The sensing module and/or the section of the wall defining the port may comprise a sealing structure. The sealing structure may seal the gases flow passage from the atmosphere surrounding the port.

The sensing module may be removable from the gas delivery system. The gas delivery system may comprise a user interface. The user interface may be configured to communicate data recorded by the sensing module to a user. The user may be a patient. The user interface may comprise a display or a speaker to communicate visual or audial data to a user.

The modulation section may comprise a converging-diverging section or a diverging-converging section. A converging portion of the converging-diverging section can comprise a curved wall surface and a diverging portion of the converging-diverging section can comprise a frustoconical surface. The gas delivery system may comprise a flow sensor. The flow sensor may be configured to measure a gases flow rate of gases at or near the first region. The flow sensor may be a component of the sensing module. The gas delivery system may be configured to calculate the dynamic pressure of gases at or near the first region. The calculation may be based on data recorded by the flow sensor. The gas delivery system may be configured to add the dynamic pressure calculated to the measured pressure of gases at or near the first region to obtain a total pressure value. The gas delivery system may comprise a user interface that can communicate the total pressure value to a user. The user may be a patient. The user interface may comprise a display or a speaker to communicate the total pressure value visually or audially. In some configurations, if the total pressure value is greater than or equal to a threshold pressure value, the gas delivery system may be reconfigured such that the total pressure of gases at or near the first region is decreased. For example, a controller of the gas delivery system may adjust the flow generator to decrease the total pressure of gases at or near the first region. In some configurations, if the total pressure value is greater than or equal to a threshold pressure value, the gas delivery system may generate an alert. The alert may be communicated to a user or patient through a user interface. The alert may comprise an audial or visual signal. The threshold pressure value in each case may be a predetermined value or may be a function of a variable measured by the sensing module or another variable. The function may be, for example, a function of the tidal volume of a patient, a function of the height of a patient, a function of the weight of a patient, a function of the measured flow rate, a function of the pressure of gases passing through the first region, or a function of some other variable.

Additionally, in accordance with at least one of the embodiments disclosed herein, method of measuring a gas pressure is disclosed. The method of measuring a gas pressure may be part of a method of providing respiratory therapy. Gas may be propelled through a gases flow passage comprising a modulation section. The velocity of gases passing through the modulation section may be modulated. In some configurations, the velocity of such gases may increase. In some configurations, the velocity of such gases may decrease. The modulation section may comprise a first region at which the modulation of the velocity of the gases is greatest or at a maximum. The pressure of gases at or near the first region may be measured. The gas may be allowed to exit an unsealed patient interface. The gases flow passage may be configured such that the measured pressure of gases at or near the first region may be substantially the same as the static pressure of gases at or near a second region along the gases flow passage downstream of the first region.

The measured pressure may be communicated to a user. The user may be a patient. The communication may occur through a user interface. The user interface may comprise a display or speaker capable of relaying visual or audial information. The gas flow rate of gases at or near the first region may be measured. The dynamic pressure of gases moving at or near the first region may be calculated. The calculation may be based on the measured gas flow rate of gases at or near the first region. The dynamic pressure calculated may be added to the measured pressure of gases at or near the first region to obtain a total pressure value. The total pressure value may be communicated to the user. In some configurations, if the total pressure value is greater than or equal to a threshold pressure value, the total pressure of gases at or near the first section may be reduced. In some configurations, if the total pressure value is greater than or equal to a threshold pressure value, an alert may be communicated to the user. The threshold pressure value in each case may be a predetermined value or may be a function of a variable measured by the sensing module or another variable. The function may be, for example, a function of the tidal volume of a patient, a function of the height of a patient, a function of the weight of a patient, a function of the measured flow rate, a function of the pressure of gases passing through the first region, or a function of some other variable.

Additionally, in accordance with at least one of the embodiments disclosed herein, an interface is disclosed. The interface may comprise a nasal cannula. The interface may comprise a gases flow passage. The gases flow passage may be adapted to receive gases from a flow generator and channel them to a patient. The gases flow passage may comprise a modulation section over which the velocity of gases passing through the modulation section is modulated. In some configurations, the velocity of such gases may increase. In some configurations, the velocity of such gases may decrease. The modulation section may comprise a first region at which the modulation of the velocity of gases passing through the modulation section is greatest or at a maximum. The gases flow passage may comprise or be configured to interface with a sensing module configured to measure the pressure of gases at or near the first region. The gases flow passage may comprise a wall. The wall may comprise a port. The port may be located at a part of the wall at or near the first region. The port may be sealed from the atmosphere outside of the port. The sensing module may be located in or on the port. The sensing module and/or the section of the wall defining the port may comprise a sealing structure. The sealing structure may seal the gases flow passage from the atmosphere surrounding the port. The gases flow passage may be configured such that, when the flow generator is propelling gases through the gases flow passage, the measured pressure of gases at or near the first region may be substantially the same as the static pressure of gases at or near a second region along the gases flow passage downstream of the first region.

The modulation section of the nasal cannula can comprise a converging-diverging section or a diverging-converging section. A converging portion of the converging- diverging section can comprise a curved wall surface and a diverging portion of the converging-diverging section comprises a frustoconical surface. A portion of the gases flow passage including the converging-diverging section can be integrated with the nasal cannula. A length of a converging portion of the converging-diverging section and a length of a diverging portion of the converging-diverging section can be different from one another.

Any one or more of the embodiments or configurations as disclosed herein may be provided in combination with any other of the one or more embodiments or configurations.

### BRIEF DESCRIPTION OF THE DRAWINGS

Throughout the drawings, reference numbers can be reused or be changed at the leading numeral(s) to indicate general correspondence between reference elements. The drawings are provided to illustrate example embodiments described herein and are not intended to limit the scope of the disclosure.

FIG. 1 shows a schematic diagram of a respiratory therapy system.

FIG. 2A illustrates a section of a gases flow passage of gas delivery system comprising a converging-diverging modulation section.

FIG. 2B illustrates a diagram of a section of a gases flow passage of a gas delivery system comprising a Venturi channel.

FIG. 2C illustrates a section of a gases flow passage of a gas delivery system comprising a diverging-converging modulation section.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

As noted above, in some cases it is useful to find a configuration for a sensing arrangement of a respiratory therapy system that can permit a pressure measurement at or near the location where gases enter the airway of a patient while also allowing the placement of the pressure sensor that obtains the pressure measurement away from the location at or near where gases enter the airway of a patient. An aspect of at least one of the configurations disclosed herein includes the realization that pressure readings of gases at a second location along the gases flow passage of a gas delivery system may be obtained at a first location along the gases flow passage if the gases flow passage comprises a modulation section along the gases flow passage and the dimensions of the gases flow passage are carefully chosen.

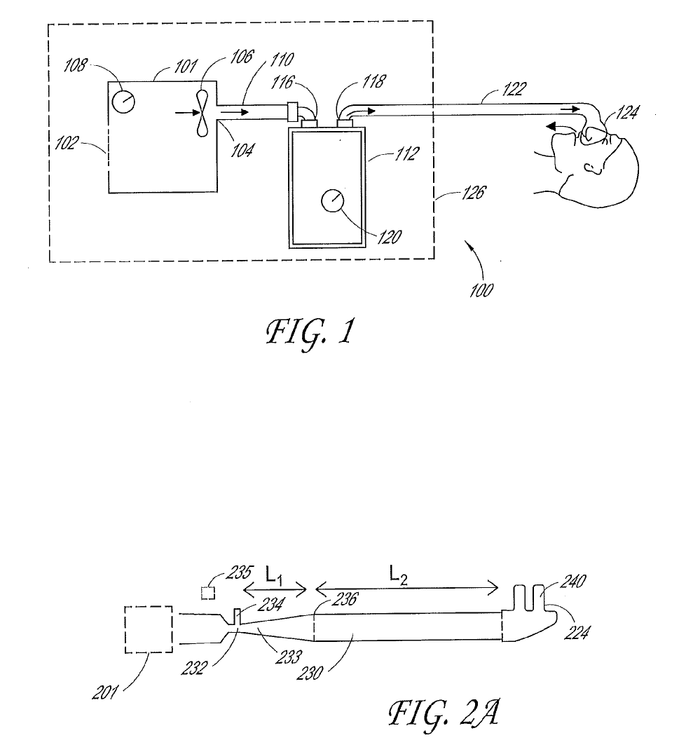

With reference to FIG. 1, a configuration for a respiratory therapy system 100 is shown. In the illustrated configuration, the respiratory system 100 may comprise a flow generator 101. The flow generator 101 may comprise a gas inlet 102 and a gas outlet 104. The flow generator may comprise a blower 106. The blower 106 may comprise a motor. The motor may comprise a stator and a rotor. The rotor may comprise a shaft. An impeller may be linked to the shaft. In use, the impeller may rotate concurrently with the shaft to draw in gas from the gas inlet 102. The flow generator 101 may comprise a user interface 108, which may comprise one or more buttons, knobs, dials, switches, levers, touch screens, and/or displays so that a user might input operation parameters into the flow generator 101 to control its operation or operation of other aspects of the respiratory therapy system 100. The flow generator 101 may pass gas through the gas outlet 104 to a first conduit 110. The first conduit 110 may pass the gas to a humidifier 112 that may entrain moisture in the gas to provide a humidified gas stream. The humidifier 112 may comprise a humidifier inlet 116 and a humidifier outlet 118. The humidifier 112 may comprise water or another moisturizing agent (hereinafter referred to as water). The humidifier 112 may comprise a heating element. The heating element may be used to heat the water in the humidifier 112 to encourage water vaporization and/or entrainment in the gas flow and/or increase the temperature and/or humidity of gases passing through the humidifier 112. The humidifier 112 may comprise a user interface 120, which may comprise one or more buttons, knobs, dials, switches, levers, touch screens, and/or displays so that a user might input operation parameters into the humidifier 112 to control the operation of the heating element, operation of other aspects of the humidifier 112, and/or other aspects of the respiratory therapy system 100. Gas may then pass from the humidifier outlet 118 to a second conduit 122. The second conduit 122 may comprise a heater. The heater may be used to add heat to gases passing through the second conduit 122. The heat may reduce or eliminate the likelihood of condensation of water entrained in the gas stream along the walls of the second conduit 122. The heater may comprise one or more resistive wires located in, on, around or near the walls of the second conduit 122. Gas passing through the second conduit 122 may then enter a patient interface 124 that may pneumatically link the respiratory therapy system 100 to the patient's airway. The patient interface 124 may comprise a nasal mask, an oral mask, an oro-nasal mask, a full face mask, a nasal pillows mask, a nasal cannula, an endotracheal tube, a combination of the above or some other gas conveying system. In one configuration, the patient interface 124 is an unsealed interface in which the interface intentionally does not create a complete seal with the user's face.

In the illustrated configuration, and as implied above, the respiratory therapy system 100 may operate as follows. Gas may be drawn into the flow generator 101 through the gas inlet 102 due to the rotation of an impeller of the motor of the blower 106. Gas may then be propelled out of the gas outlet 104 and along the first conduit 110. The gas flow may enter the humidifier 112 through the humidifier inlet 116. Once in the humidifier 112, the gas may pick up moisture. The water in the humidifier 112 may be heated by the heating element, which may aid in the humidification and/or heating of the gas passing through the humidifier 112. The gas may then leave the humidifier 112 through the humidifier outlet 118 and enter the second conduit 122. Gas may then be passed from the second conduit 122 to the patient interface 124, where it may be taken into the patient's airways to aid in the treatment of respiratory disorders.

It should be understood that the illustrated configuration should not be taken to be limiting, and that many other configurations for the respiratory therapy system 100 are possible. In some configurations, the flow generator 101 may, for example, comprise a source or container of compressed gas (e.g. air). The container may comprise a valve that may be adjusted to control the flow of gas leaving the container. In some configurations, the flow generator 101 may use such a source of compressed gas and/or another gas source in lieu of a blower 106. In some configurations, the blower 106 may be used in conjunction with another gas source. In some configurations, the flow generator 101 may draw in atmospheric gases through the gas inlet 102. In some configurations, the flow generator 101 may be adapted to both draw in atmospheric gases through the gas inlet 102 and accept other gases (e.g. oxygen, nitric oxide, carbon dioxide, etc.) through the same gas inlet 102 or a different inlet. In some configurations, the humidifier 112 can be integrated with the flow generator 101. In some configurations, the humidifier 112 and the flow generator 101 may share a housing 126. In some such configurations, only a single conduit extending between the flow generator 101 and the patient interface 124 or between the humidifier 112 and the patient interface 124 is used to convey gases to a patient. In some configurations, the flow generator 101 and the humidifier 112 may have a single user interface located on either the flow generator 101 or the humidifier 112. In some configurations, the operation of the flow generator 101, of the humidifier 112, or of other aspects of the respiratory therapy system 100 may be controlled by a controller. The controller may comprise a microprocessor. The controller may be located in or on the flow generator 101, the humidifier 112, or other parts of the respiratory therapy system 100. In some configurations, multiple controllers may be used. In some configurations, the operation of the flow generator 101, of the humidifier 112, or of other aspects of the respiratory therapy system 100 may be controlled wirelessly using a user interface located on a remote computing device. In some configurations, the respiratory therapy system 100 may comprise one or more sensors for detecting various characteristics of the gas, including pressure, flow rate, temperature, absolute humidity, relative humidity, enthalpy, oxygen concentration, and/or carbon dioxide concentration. In some configurations, there may be no user interface or a minimal user interface for the flow generator 101, humidifier 112, or other aspects of the respiratory therapy system 100. In some such configurations, the respiratory therapy system 100 may utilize a sensor to determine if the patient is attempting to use the respiratory therapy system 100 and automatically operate (e.g. the flow generator 101 may propel gases, the humidifier 112 may humidify gases, etc.) according to one or more predetermined parameters if the sensor indicates that the patient is attempting to use the respiratory therapy system 100.

Attention is now given to the use of a gas delivery system. In some cases, a gas delivery system may be a respiratory therapy system adapted to deliver gases to a patient similar to that described above. However, it should be understood that the gas delivery system may simply be understood as a gases flow generating system that may channel gases to a subject. The subject may be a patient, a room, a container, or other object. The following disclosure involves the understanding of a gas delivery system to be a respiratory therapy system and understanding of the subject to be a patient, but it should be understood that such disclosure and understandings are not to be taken as limiting. When utilizing the gas delivery system to treat a patient with gases, if the pressure of gases at or near the airway of the patient can be measured, the patient or another user or administrator of the gas delivery system may be alerted or the gas delivery system may recommend a pressure adjustment or automatically reconfigure the gas delivery system such that the pressure of gases delivered to the patient may be reduced. It is possible to configure a gases flow passage of the gas delivery system such that the gases flow passage comprises a modulation section. In some configurations, the modulation section may be placed at or close to a part of a patient interface such as a nasal cannula. For example, the modulation section may be placed at or close to a prong or manifold of a nasal cannula. In some configurations, the modulation section may comprise a section with a diameter that is less than the diameter of other portions of the gases flow passage. For example, the modulation section may comprise a converging-diverging section, such as a Venturi tube section or an orifice plate section. In some configurations, the modulation section may comprise a section with a diameter that is greater than the diameter of other portions of the gases flow passage. For example, the modulation section may comprise a diverging-converging section. The gases flow passage may be configured with specialized dimensions and proportions such that the modulation section enables a pressure value of a gas flow at a second location along the gases flow passage to be measured or estimated by finding the pressure of the gas flow at a first location. This concept is further described using the following examples.

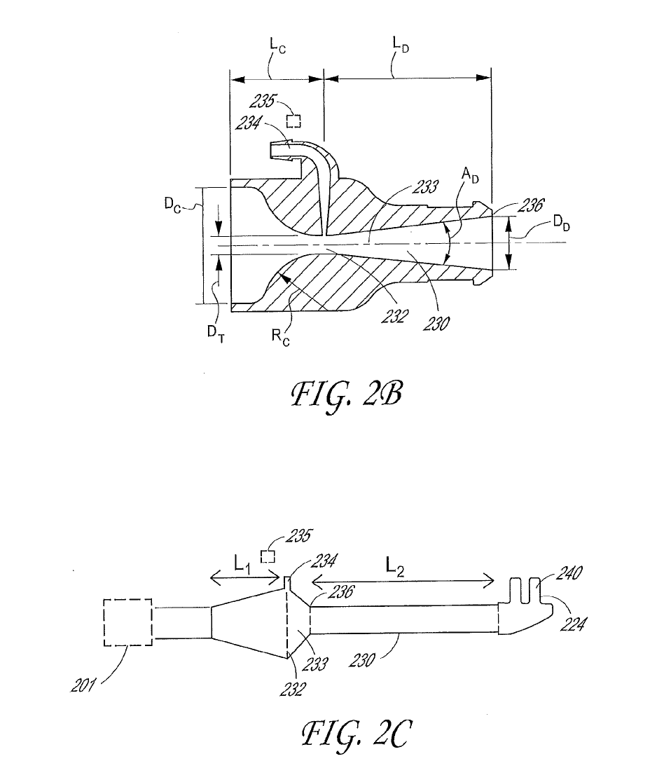

With reference to FIG. 2A, a configuration of a portion of a gases flow passage 230 of a gas delivery system is shown. The system shown in FIGS. 2A-2C can be the same as or similar to the system of FIG. 1 except as otherwise indicated herein. In general, the same or corresponding components are indicated by the same reference numbers, with the exception of the first or leading digit of the reference numbers. In FIG. 1, the reference numbers begin with the numeral “1” and, in FIGS. 2A-2C, the reference numbers begin with the numeral “2.” The illustrated gases flow passage 230 is well-suited for use with a non-sealing patient interface, such as a nasal cannula, in which the interface does not completely seal with the user's face and is described in connection with such a system. However, the illustrated gases flow passage 230 or portions thereof (e.g., modulation section 233) could be used in or adapted for use in sealed interface systems, as well.

The gases flow passage 230 may comprise a modulation section 233. The modulation section 233 may modulate the velocity or flow rate of gases passing through the modulation section 233. For example, if the modulation section 233 has a diameter that is less than the diameter of sections of the gases flow passage 230 outside of the modulation section 233, as illustrated in FIG. 2A, the modulation section 233 may increase the velocity of gases passing through the modulation section 233. If the modulation section 233 has a diameter that is greater than the diameter of sections of the gases flow passage 230 outside of the modulation section 233, the modulation section 233 may decrease the velocity of gases passing through the modulation section 233. The modulation section 233 may comprise a region 232 at or over which the modulation of the velocity of gases passing through the modulation section 233 is greatest or at a maximum (e.g., where the velocity of gases is highest or lowest relative to one or more sections of the gases flow passage 230 outside of the modulation section 233). The region 232 can be a system maximum (e.g., where the velocity of gases is highest or lowest relative to a velocity anywhere else within the system) or can be a localized maximum. In some configurations, the region 232 is a maximum within a final breathing circuit portion of system, which can be the entire breathing circuit downstream (or proximate the user) from the last principal or significant system component (e.g., the flow generator 201 or optional humidifier). In some configurations, the region 232 is a maximum within the portion of the gases flow passage 230 between the modulation section 233 and the user interface (e.g., nasal cannula) 224.

In some configurations, the illustrated gases flow passage 230 comprises a breathing circuit having at least a first portion or component and a second portion or component. The first portion or component can be a gases conduit or tube that is separate from the second portion or component. The second portion or component can be a gases conduit or tube that is integrated with the patient interface 224. For example, the patient interface 224 can be a nasal cannula that can include an integrated gases conduit. The integrated conduit of the nasal cannula can be relatively short in comparison to the overall gases flow passage 230. For example, the integrated conduit of the nasal cannula can be within the range of 20-60 cm. The integrated conduit of the nasal cannula can be connected to one or more other gases conduits (e.g., the first portion or component), which connect the integrated gases conduit of the nasal cannula to other portions of the gases flow passage 230 or system (e.g., the flow generator 201 or optional humidifier). In some configurations, the modulation section 233 is partially or completely provided in the integrated gases conduit of the nasal cannula. Such an arrangement allows the maximum region 232 of the modulation section 233 to be located relatively close to, but spaced from, an outlet of the gases flow passage 230 (e.g., outlet openings of the nasal prong(s) of the nasal cannula). Placing the modulation section 233 or the region 323 thereof closer to the user or patient allows the constriction or throttle diameter (or cross-sectional area) to be larger and less obstructive to gas flow relative to a throttle placed further from the user or patient. In some configurations, all or part of the modulation section 233 is located in the gases conduit upstream (e.g., immediately upstream) of the nasal cannula/patient interface or in a component located between the upstream gases conduit and the nasal cannula/patient interface.

A sensing module 235 may obtain data related to the characteristics of gases passing through the region 232. The sensing module may obtain, for example, the pressure, flow rate, temperature, absolute humidity, relative humidity, enthalpy, oxygen content, and/or carbon dioxide content of gases passing through the region 232. In some preferred configurations, the sensing module 235 may be configured to measure the pressure of gases passing through the region 232. In some configurations, the sensing module 235 may be physically located at or near the region 232. In some configurations, the sensing module 235 may be remote from the region 232. In some configurations, the sensing module 235 may interface with a port 234 coupled to or extending from a wall of the gases flow passage 230 that is sealed from the environment outside of the port 234. In some cases, the sensing module 235 and/or the section of the wall that defines the port 234 may comprise a sealing structure that may help to seal the port 234 from the environment outside of the port 234. The gases flow passage 230 may extend between a flow generator 201 and a user interface 224. The user interface 224 may be a nasal cannula comprising a prong capable of interfacing with a nare of a patient. In the illustrated configuration of FIG. 2A, the modulation section 233 may be a converging-diverging or Venturi tube section in-line with a flow generator 201 and a user interface 224. The modulation section 233 may be of a length L1 from the region 232 to the end 236 of the modulation section 233 and the gases flow passage 230 may extend for another length L2 from the end 236 before interfacing with a patient interface 224.

According to Bernoulli's equation, under certain conditions for a particular flow, the sum of the static pressure Ps and the dynamic pressure Pd of a flow (or, more simply, the total pressure Pt of the flow) is equal to a constant throughout the flow. The dynamic pressure of the flow is equal to 0.5 times the density of the flow times the velocity of the flow squared (Pd=0.5ρV²). Stated in another way, for points 1 and 2, the following equation should hold true under certain conditions:

Ps_1+0.5ρV1²=Ps_2+0.5ρV2² Equation (1)

where Ps_1 equals the static pressure of the flow at point 1, V1 equals the velocity (flow rate) of the flow at point 1, Ps_2 equals the static pressure of the flow at point 2, and V2 equals the velocity (flow rate) of the flow at point 2. For points 1 and 2, this equation is fully valid only if the following conditions are fulfilled:

the flow has no or negligible viscosity,

the flow is steady,

the flow is incompressible,

there is no heat addition to the flow, and

there is no or negligible change in height between points 1 and 2.

Although there is no real system for which Bernoulli's equation will hold exactly true for points 1 and 2 at a given point in time, if the static pressure of a flow at point 1 is known, Bernoulli's equation can still be useful for approximating the static pressure at point 2.

Referring back to FIG. 2A, the static pressure P1 of the gases flow at or near the region 232 of the modulation section 233 of the gases flow passage 230 (hereinafter referred to as the first region 232) may be substantially related to the static pressure P2 of the gases flow at or near the second region 240 of the gases flow passage 230 by the form of the Bernoulli equation described above:

Ps_1+0.5ρV1²=Ps_2+0.5ρV2² Equation (1)

Thus, in some configurations, if the gases flow passage 230 is configured with particular or specialized dimensions, lengths, and/or angles, the static pressure Ps_1 of the gases flow at or near the first region 232 of the modulation section 233, if measured (for example, via use of the sensing module 235), may be found to be roughly equivalent to the static pressure Ps_2 of the gases flow at or near the second region 240 of the gases flow passage 230 (which, as shown in FIG. 2A, for example, may be located in or near a nasal cannula or other interface 224). Advantageously, in such configurations the static pressure Ps_2 of the gases flow at or near the second region 240 of the gases flow passage 230 may be estimated by obtaining the static pressure Ps_1 at or near the first region 232 of the modulation section 233, which may be distal from the second region 240 from the perspective of the user or patient. That is, the second region 240 can be closer to the user or patient than the first region 232.

In the illustrated configuration described above, the physical dimensions of the gases flow passage 230 (for example, as shown in FIG. 2A) may be experimentally determined by manufacturing several modulation sections for a given cannula and testing the modulation sections with, for example, a given nasal cannula. Under a given flow rate, static pressure readings of gases at the first region 232 of the modulation section 233 (obtained through the use of a first pressure sensor or some other means) may be compared to pressure readings of gases at a second region 240 of the gases flow passage 230 (for example, at a location in a nasal cannula, such as a prong or manifold of the nasal cannula) (obtained through the use of a second pressure sensor or some other means). For example, during development and internal testing, a Venturi tube section with shapes as seen in FIG. 2B and dimensions described below (units given in millimeters) was constructed for use with an Optiflow™ pediatric nasal cannula (marketed by Fisher and Paykel Healthcare) in order to assemble a gases flow passage 230 in which, in use, the static pressure Ps_1 of gases at the first region 232 of the Venturi tube section could substantially be equated to the static pressure Ps_2 of gases at a second region 240 downstream in the nasal cannula. Table 1 below demonstrates the dimensions of the gases flow passage 230 used, although it should be understood that similar gases flow passages 230 may be constructed for a variety of interfaces including nasal cannula:

TABLE 1
A list of approximate gases flow path dimensions
for the OptiflowTM pediatric nasal cannula

| | | Length of | Diameter of |
|---|---|---|---|
| | | tubing from end 236 | gases flow passage |
| | | of Venturi tube | 230 from end of |
| | Venturi | section (modulation | Venturi tube section |
| | tube | section 233) to | (modulation section |
| | section - | cannula pressure | 233) to cannula |
| OptiflowTM | neck (first | measurement | pressure measurement |
| cannula | region 232) | point (second | point (second |
| type | diameter | region 240) | region 240) |
| OptiflowTM | 3.5 mm | 320 mm | 5.0 mm |
| pediatric | | | |
| nasal | | | |
| cannula | | | |

As observed, in the illustrated configuration, only the static pressure Ps_2 of gases at the second region 240 of the gases flow passage 230 is obtained through the measurement of the pressure Ps_1 of gases at the first region 232 of the modulation section 233 of the gases flow passage 230. To calculate the total pressure Pt_2 of gases at the second region 240, the dynamic pressure Pd_2 of gases at the second region 240 can be found. In some configurations, the dynamic pressure Pd_2 of gases at the second region 240 may be found by changing the diameter (e.g., increasing the diameter) of the first region 232 of the modulation section 233 such that the pressure P1 of gases measured at the first region 232 of the modulation section 233 is higher than the pressure P2 measured at the second region 240 by an amount that approximates the dynamic pressure component Pd_2, thus allowing the total pressure Pt_2 of the second region 240 to be measured at the first region 232. In some configurations, an approximation of the dynamic pressure component Pd_2 may be calculated from data obtained from a flow sensor or some other sensor that can obtain flow rate data at or near the first region 232. The flow sensor may be located at or near the first region or at or near the port 234, or may be a component of the sensing module 235.

The illustrated modulation section 233 comprises an asymmetrical converging-diverging arrangement. In other words, a diameter (or cross-sectional area) and/or a length of the converging and diverging portions can be different from one another. In the illustrated arrangement, the converging section has a larger diameter (or cross-sectional area) and smaller length than the diverging section. The maximum diameter DC (or cross-sectional area) of the converging section can be within the range of 3-7 times, 4-6 times or about 5.25-5.75 times the minimum diameter DT (or cross-sectional area) of the region 232. In some configurations, the maximum diameter DC is between 15-25 mm or 18-22 mm, or is about 19.5 or 20 mm. In some configurations, the minimum diameter DT is between 1-10 mm or 2-5 mm, or is about 3.5 mm. The maximum diameter DD (or cross-sectional area) of the diverging section can be within the range of 1.5-4 times, 2-3 times or about 2.25-2.75 times the minimum diameter DT (or cross-sectional area) of the region 232. In some configurations, the maximum diameter DD is between 5-15 mm or 7-11 mm, or is about 9 mm. In some configurations, a length LD of the diverging section can be within a range of 1.25-3 times, 1.5-2 times or 1.75 times a length LC of the converging section. In some configurations, the length LC is between 5-25 mm or 10-20 mm, or is 15 or 16 mm and the length LD is between 20-40 mm, 25-35 mm, 25-30 mm, or is 28 mm. In some configurations, the converging section comprises a wall having a curved (e.g., convex) profile or shape. A radius RC of the curved converging section can be within a range of 2-4 times, 2.5-3.5 times or 2.75-3 times the diameter of the minimum diameter DT (or throttle) of the region 232. The radius RC of the curved converging section can be within the range of one-quarter to three-quarters of, or one-half of, the maximum diameter DC of the converging section. The radius RC can be between 5-15 mm or 8-12 mm, or can be 10 mm. The diverging section comprises a wall having a frustoconical profile or shape having an angle AD, which can be within a range of 5-25 degrees, 10-15 degrees, 11-13 degrees. In some configurations, the angle AD is 12 degrees. Such an arrangement has been determined to provide good results in connection with a nasal cannula.

Although the above paragraphs have focused on aspects of the disclosure embodied by the systems illustrated in FIGS. 2A and 2B, it should be understood that similar gases flow passages 230 may be constructed for other pressure measurement systems or gas delivery systems. For example, although the above paragraphs have focused on aspects of the disclosure involving use of a converging-diverging section or a Venturi tube section in such gases flow passages 230, in some configurations, and as seen in FIG. 2C, a diverging-converging section or ‘reverse Venturi’ tube section may be used in the gases flow passage 230. As demonstrated in the above paragraphs, the knowledge gained by studying Bernoulli's equation may similarly be applied by experimentally or theoretically finding dimensions of the components of the gases flow passage 230 such that the static pressure Ps_1 at the first region 232 of the gases flow passage 230, if measured, may be equivalent to the static pressure Ps_2 at a second region 240 of the gases flow passage 230, which may be downstream of the first region 232. It should also be understood that other modulation sections 233 may be constructed for other gases flow passages 230. The other modulation sections 233 may have other shapes, e.g., converging-diverging-converging-diverging shapes, parabolic shapes, and so on. The particular shape and dimension of the modulation section 233 for any particular product(s) (e.g., patient interface, breathing circuit or modulation component) that incorporates a flow passage 230 can be determined experimentally, using a second pressure and/or flow sensor at the second point of interest, as described herein. However, the modulation section 233 may also be configured theoretically or by extrapolating experimental results to apply to similar product(s). For example, the shape and/or size of the modulation section 233 can be calculated in view of differences between a characteristic (e.g., length, diameter or other dimension) in the experimental product and the product being designed.

Once the total pressure of gases at or near the second region 240 is found, the total pressure may be relayed to a user of the system, a patient, or another subject through a user interface of the gas delivery system. The user interface may be a part of the gas delivery system or may be remote from the gas delivery system. In some configurations, the total pressure may be compared to a threshold pressure. The threshold pressure may be a predetermined pressure or may be a function of parameters measured by a sensing module (for example, the sensing module 235) of the gas delivery system. In some configurations, if the total pressure of gases at or near the second region 240 is greater than or equal to the threshold pressure, the user interface may alert the user through an output module capable of delivering audial output, visual output, tactile output, olfactory output, or other forms of output. In some configurations, if the total pressure of gases at or near the second region 240 is greater than or equal to the threshold pressure, the gas delivery system may be reconfigured such that the total pressure of gases at or near the second region 240 may be reduced. For example, a blower or flow generator of the gas delivery system may be adjusted, or a valve in-line between the flow generator of the gas delivery system and the second region 240 or the patient interface may be adjusted.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to.”

Where, in the foregoing description reference has been made to integers or components having known equivalents thereof, those integers are herein incorporated as if individually set forth.

The invention may also be said broadly to consist in the parts, elements and features referred to or indicated in the specification of the application, individually or collectively, in any or all combinations of two or more of said parts, elements or features.

Reference to any prior art in this specification is not, and should not be taken as, an acknowledgement or any form of suggestion that that prior art forms part of the common general knowledge in the field of endeavour in any country in the world.

Certain features, aspects and advantages of some configurations of the present disclosure have been described with reference to use of the gas delivery system as a respiratory therapy system. However, certain features, aspects and advantages of the use of the gas delivery system as described may be advantageously utilized in other applications involving gas flows, including technologies involving hydrodynamics and aerodynamics. Additionally, certain features, aspects and advantages of the gas delivery system may be applied to general fluid delivery systems adapted to deliver fluids (which may include flowing solids, liquids, and/or gases) to a subject. In summary, certain features, aspects and advantages of the methods and apparatus of the present disclosure may be equally applied to other applications. It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

1. A gas delivery system comprising:
a flow generator adapted to propel gases;
a gases flow passage adapted to receive gases from the flow generator and channel them to a subject;
an unsealed patient interface; and
a sensing module configured to measure a gas pressure;
wherein the gases flow passage comprises a modulation section where the velocity of gases passing through the modulation section is modulated, the modulation section comprising a first region at which the modulation of the velocity of the gases is greatest;
wherein the sensing module measures the pressure of gases at or near the first region; and
wherein the gases flow passage is configured such that, when the flow generator is propelling gases through the gases flow passage, the measured pressure of gases at or near the first region is substantially the same as the static pressure of gases at or near a second region along the gases flow passage downstream of the first region.

2. The gas delivery system of claim 1, wherein the gases flow passage comprises a wall, the wall comprising a port located at a part of the wall at or near the first region, the port being sealed from the atmosphere outside of the port.

3. The gas delivery system of claim 1, further comprising a user interface configured to communicate the data recorded by the sensing module to a user.

4. The gas delivery system of claim 1, wherein the modulation section comprises a converging-diverging section.

5. The gas delivery system of claim 4, wherein a converging portion of the converging-diverging section comprises a curved wall surface and a diverging portion of the converging-diverging section comprises a frustoconical surface.

6. The gas delivery system of claim 1, wherein the modulation section comprises a diverging-converging section.

7. The gas delivery system of claim 1, further comprising a flow sensor configured to measure a gases flow rate of gases at or near the first region.

8. The gas delivery system of claim 7, wherein the flow sensor is a component of the sensing module.

9. The gas delivery system of claim 7, wherein the gas delivery system is configured to calculate the dynamic pressure of gases at or near the first region based on data measured by the flow sensor.

10. The gas delivery system of claim 9, wherein the gas delivery system is configured to add the dynamic pressure calculated to the measured pressure of gases at or near the first region to obtain a total pressure value.

11. The gas delivery system of claim 10, wherein if the total pressure value is greater than or equal to a threshold pressure value, the gas delivery system is reconfigured such that the total pressure of gases at or near the first region is decreased.

12. The gas delivery system of claim 10, wherein if the total pressure value is greater than or equal to a threshold pressure value, the gas delivery system generates an alert.

13. A method of measuring a gas pressure comprising:
propelling a gas through a gases flow passage comprising a modulation section where the velocity of gases passing through the modulation section is modulated, the modulation section comprising a first region at which the modulation of the velocity of the gases is greatest;
measuring the pressure of gases at or near the first region; and
allowing the gas to exit an unsealed patient interface;
wherein the gases flow passage is configured such that the measured pressure of gases at or near the first region is substantially the same as the static pressure of gases at or near a second region along the gases flow passage downstream of the first region.

14. The method of claim 13, further comprising the step of measuring the gas flow rate of gases at or near the first region.

15. The method of claim 14, further comprising the step of calculating the dynamic pressure of gases at or near the first region based on the measured gas flow rate of gases at or near the first region.

16. The method of claim 15, further comprising the step of adding the calculated dynamic pressure to the measured pressure of gases at or near the first region to obtain a total pressure value.

17. The method of claim 16, wherein, if the total pressure value is greater than or equal to a threshold pressure value, the method further comprises reducing the total pressure of gases at or near the first region.

18. The method of claim 17, wherein the threshold pressure value is a predetermined value.

19. The method of claim 17, wherein the threshold pressure value is a function of the measured pressure and/or the measured flow rate.

20. The method of claim 16, wherein, if the total pressure value is greater than or equal to a threshold pressure value, the method further comprises communicating an alert to a user.
